(19) United States
(12) Patent Application Publication
(10) Pub. No.: US 2021/0383283 A1
(43) Pub. Date: Dec. 9, 2021

(54) ELECTRONIC DEVICE, COMPUTER-READABLE NON-TRANSITORY RECORDING MEDIUM STORING PHARMACY RESERVATION PROGRAM, AND PHARMACY RESERVATION SYSTEM

(71) Applicant: KYOCERA Document Solutions Inc., Osaka-shi (JP)

(72) Inventors: Daiki SONE, Oska-shi (JP); Tomoharu Tokunaga, Osaka-shi (JP); Tomoki Nakaya, Osaka-shi (JP); Kenzo Azakami, Osaka-shi (JP)

(21) Appl. No.: 17/335,157

(22) Filed: Jun. 1, 2021

(30) Foreign Application Priority Data
Jun. 3, 2020 (JP) 2020-097216

(51) Int. Cl. G06Q 10/02; H04L 29/08; G16H 20/13

(52) U.S. Cl. CPC G06Q 10/02; G16H 20/13; H04L 67/125; H04L 67/16

(57) ABSTRACT

A dispensing reservation reception system is capable of receiving dispensing reservation information sent by a medication notebook application of a user terminal, and an image forming device is capable of generating the dispensing reservation information in accordance with a specification of the medication notebook application of each of a plurality of pharmacies, and sends, to the dispensing reservation reception system of a target pharmacy, the dispensing reservation information generated in accordance with the specification of the medication notebook application of the target pharmacy as a pharmacy selected from among a plurality of pharmacies, in a sending method adapted to the specification of the medication notebook application of the target pharmacy.

### INCORPORATION BY REFERENCE

This application is based upon, and claims the benefit of priority from, corresponding Japanese Patent Application No. 2020-097216 filed in the Japan Patent Office on Jun. 3, 2020, the entire contents of which are incorporated herein by reference.

### BACKGROUND

### Field of the Invention

The present disclosure relates to an electronic device for making a dispensing reservation at a pharmacy, a computer-readable non-transitory recording medium storing pharmacy reservation program, and a pharmacy reservation system.

### Description of Related Art

A typical electronic device is known to make a dispensing reservation at the pharmacy by sending prescription data to the pharmacy in a pharmacy-specific output method such as a fax, email, or electronic prescription system, and in a pharmacy-specific output data format such as image data or PDF (Portable Document Format).

### SUMMARY

An electronic device of the present disclosure receives dispensing reservation information as information of a dispensing reservation at a pharmacy and thereby sends the dispensing reservation information to a dispensing reservation reception system that receives the dispensing reservation at the pharmacy, wherein the dispensing reservation reception system is capable of receiving the dispensing reservation information sent by a medication notebook application of a user terminal that realizes the medication notebook application as an application for sending the dispensing reservation information, and the electronic device is capable of generating the dispensing reservation information in accordance with a specification of the medication notebook application of each of a plurality of the pharmacies, and sends, to the dispensing reservation reception system of a target pharmacy, the dispensing reservation information generated in accordance with the specification of the medication notebook application of the target pharmacy as a pharmacy selected from among the plurality of the pharmacies, in a sending method adapted to the specification of the medication notebook application of the target pharmacy.

A computer-readable non-transitory recording medium of the present disclosure stores a pharmacy reservation program, and the pharmacy reservation program receives dispensing reservation information as information of a dispensing reservation at a pharmacy and thereby sends the dispensing reservation information to a dispensing reservation reception system that receives the dispensing reservation at the pharmacy. The dispensing reservation reception system is capable of receiving the dispensing reservation information sent by a medication notebook application of a user terminal that realizes the medication notebook application as an application for sending the dispensing reservation information, and the pharmacy reservation program is capable of causing an electronic device to generate the dispensing reservation information in accordance with a specification of the medication notebook application of each of a plurality of the pharmacies, and causes the electronic device to send, to the dispensing reservation reception system of a target pharmacy, the dispensing reservation information which the pharmacy reservation program caused the electronic device to generate in accordance with the specification of the medication notebook application of the target pharmacy as a pharmacy selected from among the plurality of the pharmacies, in a sending method adapted to the specification of the medication notebook application of the target pharmacy.

A pharmacy reservation system of the present disclosure receives dispensing reservation information as information of a dispensing reservation at a pharmacy and thereby sends the dispensing reservation information to a dispensing reservation reception system that receives the dispensing reservation at the pharmacy, wherein the dispensing reservation reception system is capable of receiving the dispensing reservation information sent by a medication notebook application of a user terminal that realizes the medication notebook application as an application for sending the dispensing reservation information, and the pharmacy reservation system is capable of generating the dispensing reservation information in accordance with a specification of the medication notebook application of each of a plurality of the pharmacies, and sends, to the dispensing reservation reception system of a target pharmacy, the dispensing reservation information generated in accordance with the specification of the medication notebook application of the target pharmacy as a pharmacy selected from among the plurality of pharmacies, in a sending method adapted to the specification of the medication notebook application of the target pharmacy.

### BRIEF DESCRIPTION OF THE DRAWINGS

FIG. 1 is a block diagram of a system according to an embodiment of the present disclosure; and

FIG. 2 is a block diagram of a clinic information integrated management system shown in FIG. 1 when the system is configured by a single computer; and

FIG. 3 is a block diagram of a settlement system shown in FIG. 1;

FIG. 4 is a block diagram of hardware of the image forming device shown in FIG. 1 in the case of being an MFP;

FIG. 5 is a block diagram of the control configuration of the image forming device shown in FIG. 4;

FIG. 6 is a block diagram of the user terminal shown in FIG. 1;

FIG. 7 is a flowchart of the operation of the image forming device shown in

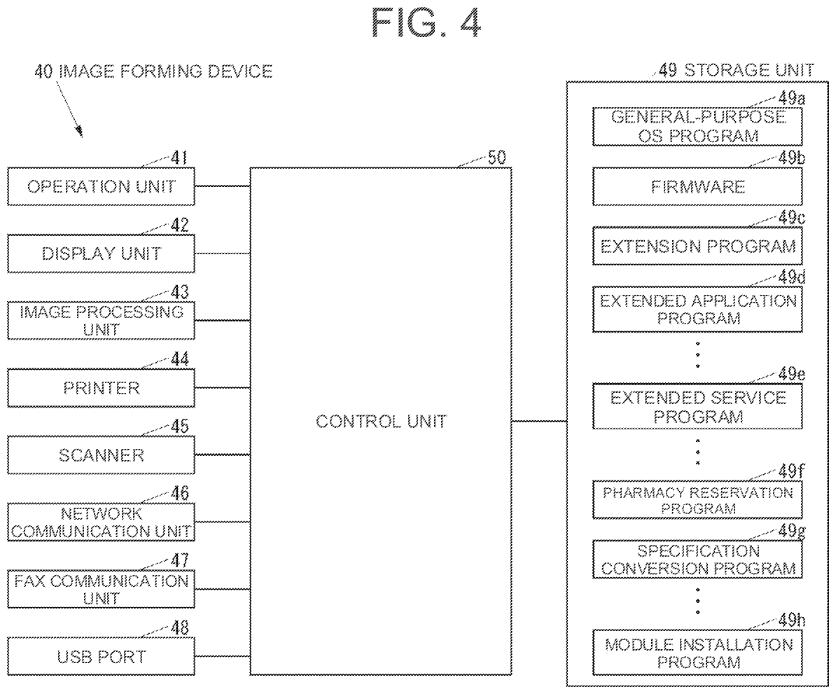

FIG. 4 when a specification conversion program is to be installed;

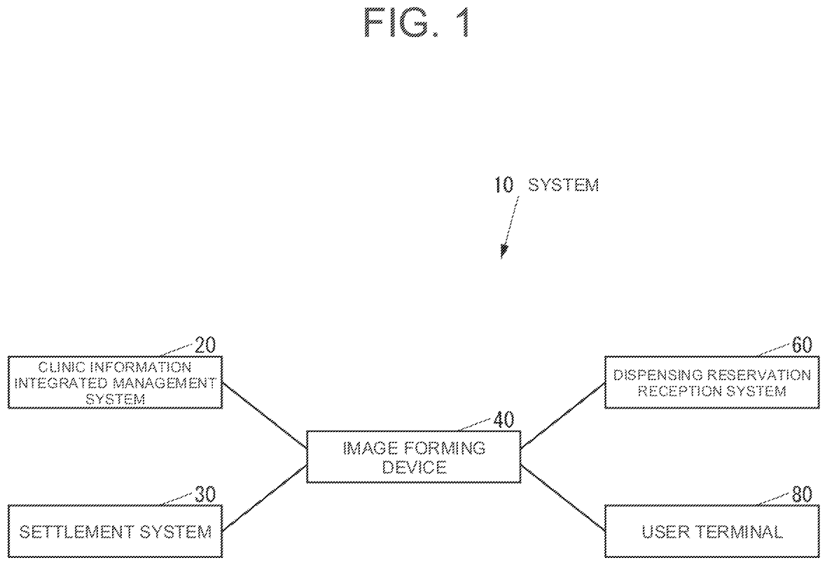

FIG. 8 is a sequence diagram of the operation of the system shown in FIG. 1 when the medical expense is collected;

FIG. 9 is a sequence diagram of “Reserved” shown in FIG. 8;

FIG. 10 is a sequence diagram of “User Terminal Module Load” shown in FIG. 9;

FIG. 11 is a sequence diagram of “Pharmacy Module Load” shown in FIG. 9;

FIG. 12 is a sequence diagram of “Not Reserved” shown in FIG. 8; and

FIG. 13 is a sequence diagram of the operation of the system shown in FIG. 1 when dispensing reservation is made by a medication notebook application.

### DETAILED DESCRIPTION

The following describes an embodiment of the present disclosure with reference to the drawings.

First, the structure of a system according to an embodiment of the present disclosure is described.

FIG. 1 is a block diagram of a system 10 according to the present system.

As shown in FIG. 1, the system 10 includes a clinic information integrated management system 20 that manages various information of a clinic, such as a hospital or a doctor's office. The clinic information integrated management system 20 may be configured by a single computer or by a plurality of computers. Other than the clinic information integration management system 20, the system 10 can include at least one clinic information integration management system similar in configuration to the clinic information integration management system 20. The system 10 has a clinic information integrated management system for each clinic.

The system 10 is installed in the clinic and has a settlement system 30, such as an automatic payment machine which collects, from patients, medical expenses such as medical fees for this clinic. Other than the settlement system 30, the system 10 can include at least one other settlement system similar in configuration to the settlement system 30. The system 10 has the settlement system for each clinic.

The system 10 has an image forming device 40, such as a multifunction peripheral, a printer-only machine, or the like, as an electronic device that is installed in the clinic and prints prescriptions in response to an instruction from the settlement system. Other than the image forming device 40, the system 10 can include at least one image forming device similar in configuration to the image forming device 40. The system 10 has the image forming device in each clinic.

The clinic information integrated management system, the settlement system, and the image forming device for the same clinic include a pharmacy reservation system of the present disclosure.

The system 10 has a dispensing reservation reception system 60 that accepts dispensing reservations to pharmacies. For receiving information on dispensing reservation at the pharmacy (hereinafter referred to as “dispensing reservation information”) from a medication notebook application described below, the dispensing reservation reception system 60 has an API (Application Programming Interface) that is called by the medication notebook application. The dispensing reservation reception system 60 may include a single computer or a plurality of computers. Other than the dispensing reservation reception system 60, the system 10 can include at least one dispensing reservation reception system similar in configuration to the dispensing reservation reception system 60. The system 10 has the dispensing reservation reception system for each pharmacy or each group of pharmacies. For each dispensing reservation reception system, the API called by the medication notebook application is different in the specification of dispensing reservation at the pharmacy, such as the specification of the dispensing reservation information. Therefore, the medication notebook application is different for each dispensing reservation reception system.

The system 10 has a user terminal 80 which is a portable electronic device such as a smartphone, for example, used by a user such as a patient. Other than the user terminal 80, the system 10 can include at least one user terminal similar in configuration to the user terminal 80. The system 10 has the user terminal for each user.

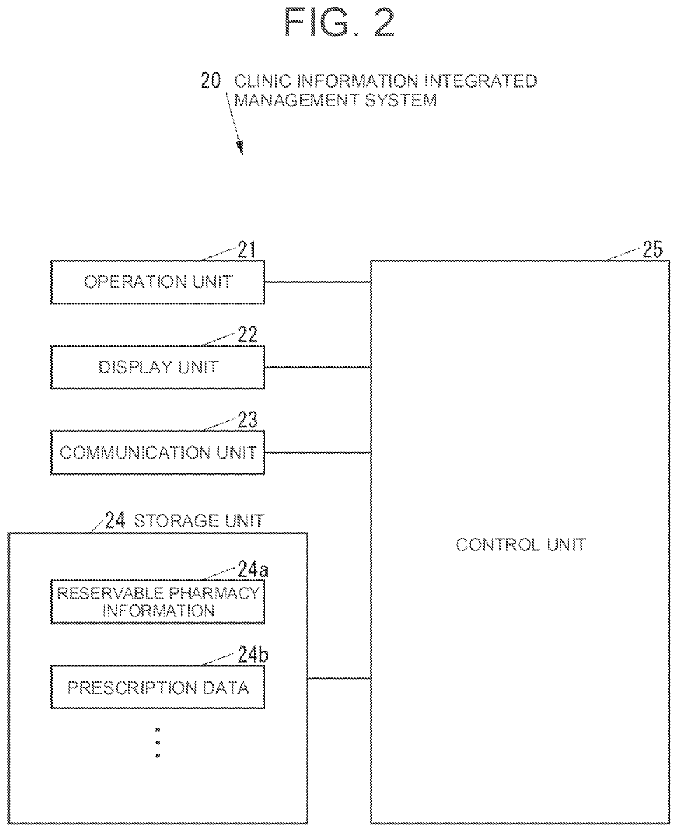

FIG. 2 is a block diagram of the clinic information integrated management system 20 when the system is configured by a single computer.

As shown in FIG. 2, the clinic information integrated management system 20 includes an operation unit 21 which is an operation device such as a button into which various operations are input, a display unit 22 which is a display device such as a liquid crystal display (LCD) for displaying various information, a communication unit 23 which is a communication device for communicating with an external device via a network such as a local area network (LAN) or the Internet or directly by wired or wireless means without going through the network, a storage unit 24 which is a non-volatile storage device such as semiconductor memory or HDD (Hard Disk Drive) that stores various information, and a control unit 25 for controlling the entire clinic information integrated management system 20.

The storage unit 24 stores information 24a on a pharmacy with which the clinic that is compatible with the clinic information integrated management system 20 is affiliated and with which it is possible to make a dispensing reservation (hereinafter referred to as “reservable pharmacy information”).

The storage unit 24 can store data of a prescription of a patient (hereinafter referred to as “prescription data”) 24b. The storage unit 24 can store at least one prescription data other than the prescription data 24b. The storage unit 24 can store prescription data for each patient.

The control unit 25 includes, for example, a CPU (Central Processing Unit), a ROM (Read Only Memory) that stores a program and various data, and a RAM (Random Access Memory) that is used as a work area for the CPU. The CPU executes the program stored in the storage unit 24 or in the ROM.

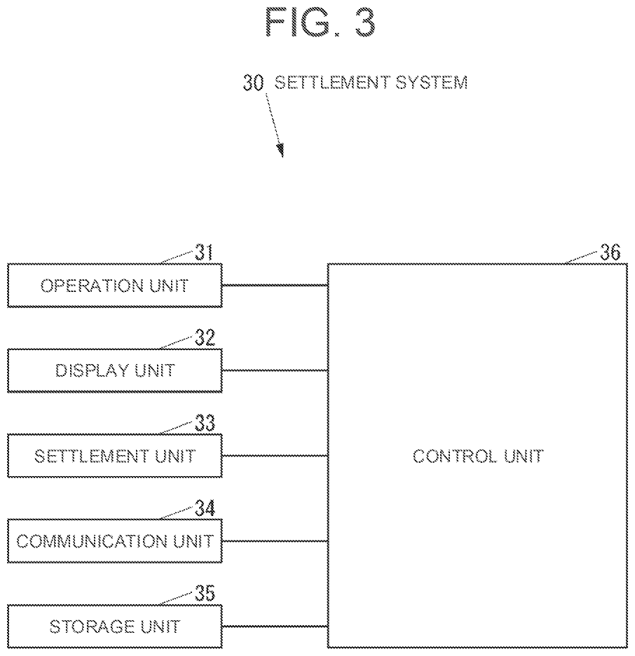

FIG. 3 is a block diagram of the settlement system 30.

As shown in FIG. 3, the settlement system 30 includes an operation unit 31 which is an operation device such as, for example, a button into which various operations are input, a display unit 32 which is a display device such as, for example, an LCD for displaying various information, a settlement unit 33 which executes settlement by money, electronic money, credit card, etc., a communication unit 34 which is a communication device that communicates with an external device via a network such as a LAN or the Internet or directly by wired or wireless means without going through the network, a storage unit 35 which is a non-volatile storage device such as a semiconductor memory or HDD that stores various information, and a control unit 36 that controls the entire settlement system 30.

The control unit 36 has, for example, a CPU, a ROM that stores a program and various data, and a RAM as a memory used as a work area of the CPU. The CPU executes the program stored in the storage unit 35 or in the ROM.

FIG. 4 is a block diagram of hardware of the image forming device 40 in the case of being an MFP.

As shown in FIG. 4, the image forming device 40 includes an operation unit 41 which is an operation device such as, for example, a button into which various operations are input, a display unit 42 which is a display device such as, for example, an LCD for displaying various information, an image processing unit 43 which is an image processing device such as, for example, a GPU (Graphics Processing Unit) for executing, on image data, various image processing such as, for example, enlargement, reduction, density adjustment, tone adjustment, image improvement, and the like, a printer 44 which is a printing device to print an image on a recording medium such as paper, a scanner 45 which is a reading device to read an image from a manuscript, a network communication unit 46 which is a communication device for communicating with an external device via a network such as a LAN or the Internet or directly by wired or wireless means without going through the network, a fax communication unit 47 which is a fax device for executing fax communication with an external facsimile device (not shown) via a communication line such as a public telephone line, a USB (Universal Serial Bus) port 48, a storage unit 49 which is a non-volatile storage device such as a semiconductor memory or HDD for storing various information, and a control unit 50 for controlling the entire image forming device 40.

The storage unit 49 stores a program 49a for a general-purpose operating system OS (hereinafter referred to as the “general-purpose OS program”) 49a such as, for example, Linux (registered trademark) for controlling each hardware of the image forming device 40. The general-purpose OS program 49a, for example, may be installed on the image forming device 40 at the manufacturing stage of the image forming device 40, may be additionally installed on the image forming device 40 from an external storage medium such as a CD (Compact Disk), DVD (Digital Versatile Disk), or USB memory (flash drive), or may be additionally installed on the image forming device 40 from the network.

The storage unit 49 stores a firmware 49b of the image forming device 40. The firmware 49b, for example, may be installed on the image forming device 40 at the manufacturing stage of the image forming device 40, may be additionally installed on the image forming device 40 from an external storage medium such as a CD, DVD, or USB memory, or may be additionally installed on the image forming device 40 from the network.

The storage unit 49 stores an extension program 49c which is a program for realizing a function other than the standard function of the image forming device 40. The extension program 49c, for example, may be installed on the image forming device 40 at the manufacturing stage of the image forming device 40, may be additionally installed on the image forming device 40 from an external storage medium such as a CD, DVD, or USB memory stick, or may be additionally installed on the image forming device 40 from the network.

The storage unit 49 stores an extended application program 49d (hereinafter referred to as “extended application program”) which is an application that realizes a function other than the standard function of the image forming device 40. The storage unit 49 can store an extended application program for each extended application. The extended application program, for example, may be installed on the image forming device 40 at the manufacturing stage of the image forming device 40, may be additionally installed on the image forming device 40 from an external storage medium such as a CD, DVD, or USB memory, or may be additionally be installed on the image forming device 40.

The storage unit 49 stores an extension service program 49e that provides a service to an extended application that uses a general-purpose OS without using a standard application which is an application that realizes the standard function of the image forming device 40 (hereinafter referred to as “extension service program”). The storage unit 49 can store the extension service program for each extension service. For example, the extension service program includes a pharmacy reservation program 49f which is an extension service program for a pharmacy reservation service which is an extension service for making a dispensing reservation at a pharmacy, a specification conversion program 49g which is an extension service program for a specification conversion module which is an extension service for converting the specification of a dispensing reservation at a pharmacy, and a module installation program 49h which is an extension service program for a module installation service which is an extension service for installing the specification conversion program. The storage unit 49 can store the specification conversion program for each type of medication notebook application described below. The extension service program, for example, may be installed on the image forming device 40 at the manufacturing stage of the image forming device 40, may be additionally installed on the image forming device 40 from an external storage medium such as a CD, DVD, or USB memory, or may be additionally installed on the image forming device 40.

The control unit 50 has, for example, a CPU, a ROM that stores a program and various data, and a RAM as a memory used as a work area of the CPU. The CPU executes a program stored in the storage unit 49 or in the ROM.

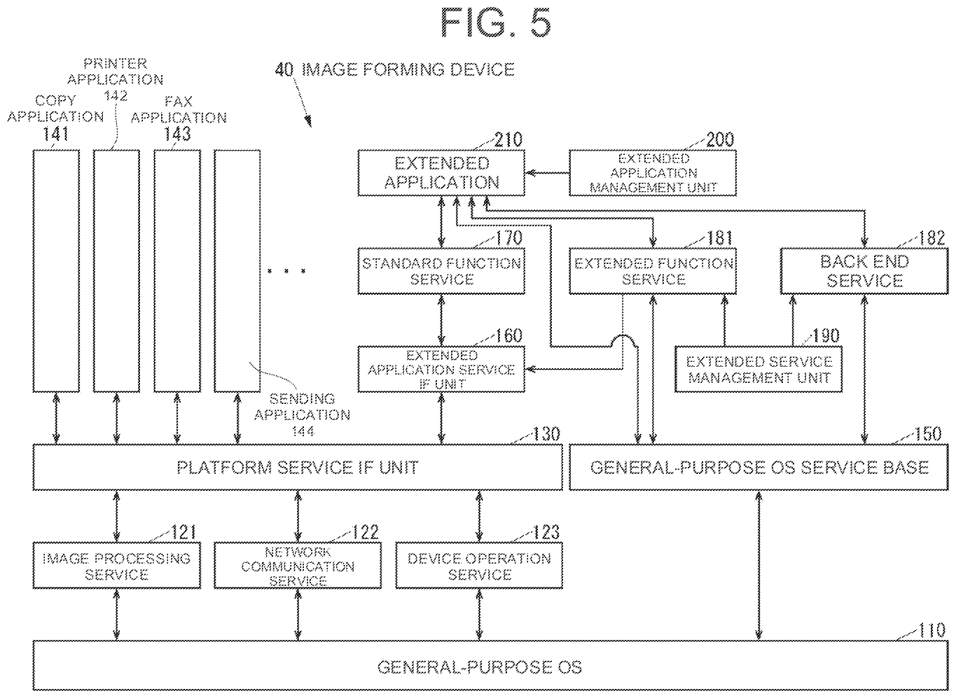

The control unit 50 realizes a control configuration in FIG. 5 by executing a program stored in the storage unit 49 or in the ROM.

FIG. 5 is a block diagram of the control configuration of the image forming device 40.

As shown in FIG. 5, the control unit 50 executes the general-purpose OS program 49a (see FIG. 4) to thereby realize a general-purpose OS 110.

By executing the firmware 49b (see FIG. 4), the control unit 50 realizes an image processing service 121 that operates on the general-purpose OS 110 and provides a service related to image processing, a network communication service 122 that operates on the general-purpose OS 110 and provides a service related to communication via a network, and a device operation service 123 that operates on the general-purpose OS 110 and provides a service related to operating each hardware of the image forming device 40. The image processing service 121, the network communication service 122, and the device operation service 123 each control respective hardware of the image forming device 40 via the general-purpose OS 110.

The image processing service 121, for example, may provide a service for storing, in the storage unit 49, image data that has been read, by the scanner 45, from a manuscript with resolution, color, and the like set, and for causing the image processing unit 43 to process the image data stored in the storage unit 49. The image processing service 121 may provide a service for drawing, based on a file, image data to be printed by the printer 44. To the image data acquired from the extended application, the image data read from the manuscript by the scanner 45, or the image data stored in the storage unit 49, the image processing service 121 may provide a service for executing image processing, such as enlargement, reduction, rotation, color conversion, noise removal, format conversion, character recognition, and watermark recognition.

By using various protocols such as TCP (Transmission Control Protocol)/IP (Internet Protocol) and UDP (User Datagram Protocol), the network communication service 122 may provide, via the network communication unit 46, a service to execute file sending/receiving, streaming, and authentication data sending/receiving. For example, the network communication service 122 can also send and receive image data read from the manuscript by the scanner 45 and can send and receive a file stored in the storage unit 49. The network communication service 122 also can send and receive the image data sent and received by the fax communication unit 47, can send and receive the result of user authentication by a user authentication unit when the image forming device 40 has the user authentication unit that authenticates the user by means of an IC (integrated circuit) card or biometric authentication, and can send and receive the image data generated by a camera when the image forming device 40 has the camera. The network communication service 122 can also send and receive information about the status of each unit of the image forming device 40 and about the result of invoking the function of each part.

The device operation service 123 may provide a service to execute processes such as drawing on the display unit 42, driving the printer 44, controlling the fax communication unit 47, authenticating users, and acquiring the status of each hardware of the image forming device 40. The device operation service 123 may provide, for example, a service to store, in the storage unit 49, the image data read from the manuscript by the scanner 45, a service for drawing an image on the browser displayed on the display unit 42, a service for sending an image data to the printer 44 for image formation, and a service for sending, by the fax communication unit 47, the image data stored in the storage unit 49. The device operation service 123, for example, when an optional device such as a finisher is present in the image forming device 40, may provide a service to cause the optional device to execute a process such as bookbinding or stapling, when the user authentication unit that authenticates the user by an IC card or biometric authentication is present in the image forming device 40, may provide a service to cause the user authentication unit to execute user authentication, and when the image forming device 40 has a camera, may provide a service to cause the camera to generate image data or to invoke functions such as eye detection which is based on the image data generated by the camera. The device operation service 123 may provide a service to execute detailed control of each hardware of the image forming device 40, such as, for example, driving one of the motors of a transport unit of the printer 44. The device operation service 123 may provide a service to acquire the status of each unit of the image forming device 40.

By executing the firmware 49b, the control unit 50 realizes a platform service IF unit 130 which provides an interface for invoking the image processing service 121, the network communication service 122, and the device operation service 123.

The control unit 50 realizes the standard application by executing the firmware 49b. The standard application runs on the platform service IF unit 130 and uses each hardware of the image forming device 40. For example, the control unit 50 realizes a copy application 141 which is a standard application that realizes copying which reads an image from a manuscript by the scanner 45 according to an instruction input to the operation unit 41 and prints the read image by the printer 44, a printer application 142 which is a standard application that prints an image which is based on data, such as data described in PDL (Page Description Language), received by the network communication unit 46, a fax application 143 which is a standard application that reads an image from a manuscript by the scanner 45 according to an instruction input to the operation unit 1 and sends the read image by the fax communication unit 47, and a sending application 144 which reads an image from a manuscript by the scanner 45 according to an instruction input to the operation unit 41 and sends the read image by the network communication unit 46. According to the instruction input to the operation unit 41, the printer application 142 may print, by the printer 44, an image which is based on a file stored in the storage unit 49. The fax application 143 may send, by the fax communication unit 47, an image which is based on a file received by the network communication unit 46, may print, by the printer 44, an image received by the fax communication unit 47, and may store, in the storage unit 49, an image received by the fax communication unit 47.

By executing the extension program 49c (see FIG. 4), the control unit 50 realizes a general-purpose OS service base 150 that provides a service using the general-purpose OS 110 not via the platform service IF unit 130. The general-purpose OS service base 150 may provide a service for adding a function to the general-purpose OS 110, such as, for example, installing control programs such as device a driver and script on the general-purpose OS 110.

By executing the extension program 49c, the control unit 50 realizes an extended application service IF unit 160 which provides, from the extended application side, an interface for using the platform service IF unit 130.

By executing the extension program 49c, the control unit 50 realizes a standard function service 170 which is a service that provides, by using the standard application, the standard function of the image forming device 40 to the extension application. The standard function service 170 uses the standard application via the extended application service IF unit 160. For example, by using the standard function service 170, the extended application can display, on the display unit 42, a screen of start of copying by the copy application 141. Further, by using the standard function service 170, the extended application can cause the printer application 142 to print various job data, files, and the like that are acquired by the extended application itself or specified via the operation unit 41. Further, by using the standard function service 170, the extended application can acquire image data, which is specified via the operation unit 41, among the image data of the facsimile received by the fax application 143, or can cause the fax application 143 to send a file or the like specified via the operation unit 41.

By executing the extension service program (see FIG. 4), the control unit 50 realizes an extension service which is a service that provides a function other than the standard function of the image forming device 40. For example, the control unit 50 realizes an extended function service 181 which is an extension service that provides the extended application with a service that uses the platform service IF unit 130 via the extended application service IF unit 160 without using the standard application or uses the general-purpose OS 110 by using the general-purpose OS service base 150, and a back end service 182 which is an extension service that provides the extended application with a service that uses the general-purpose OS 110 by using the general-purpose OS service base 150 without using the platform service IF unit 130. The control unit 50 can realize at least one extended function service other than the extended function service 181. The control unit 50 can realize at least one back end service other than the back end service 182.

The extended function service is a service that provides an extended application with the function to extend the standard function of the image forming device 40. The extended function service includes, for example, a pharmacy reservation service realized by executing the pharmacy reservation program 49f, a specification conversion module realized by executing the specification conversion program 49g, and a module installation service realized by executing the module installation program 49h.

The back end service is a service that runs on the back end. The back end service is a service that provides a function other than functions of extension of the standard function of the image forming device 40, of the functions other than the standard function of the image forming device 40. The back end service includes, for example, a service that provides a function for monitoring the image forming device 40, a service that provides a function for monitoring the entire system including a plurality of image forming devices, a service that provides a function for monitoring a specific server, a service that provides a function for collecting, from the image forming device 40, information such as a counter indicating the number of copies printed by the image forming device 40 and thereby converting the collected information into information in a format that can be understood by a human, and a service that provides a function for converting, from a format of an old interface to a format of a newer interface, a format of information output from the image forming device 40.

By executing the extension program 49c, the control unit 50 realizes an extension service management unit 190 that executes management of the extension service, such as operating, starting and stopping the extension service. The extension service management unit 190 monitors the status of the extension service and of the general-purpose OS service base 150.

By executing the extension program 49c extended application, the control unit 50 realizes an extended application management unit 200 that executes management of the extended application, such as operating, starting and stopping the extended application.

By executing the extended application program 49d (see FIG. 4), the control unit 50 realizes an extended application 210. The control unit 50 can realize at least one extended application other than the extended application 210. The extended application is an application that is used directly by the user and includes a user interface. By operating the user interface of the extended application, the user can use a function provided by this extended application. For example, the extended application may be practical applications such as OCR (Optical Character Recognition), surveillance camera application, watermark creation, invoice creation, employee list management, slip management, contract management, business card management, PDF creation, bookkeeping management, and group management, may be an installer application that makes additional changes to the device driver, firmware, etc. on the general-purpose OS 110, or may be any other management application. The extended application can invoke the standard function service 170 to thereby use the general-purpose OS 110 by using the standard application, can invoke the extension service to thereby use the general-purpose OS 110 without using the standard application, or can directly invoke the general-purpose OS service base 150 to thereby use the general-purpose OS 110.

The image forming device 40 operates as an observer of a BLE (Bluetooth (registered trademark) Low Energy).

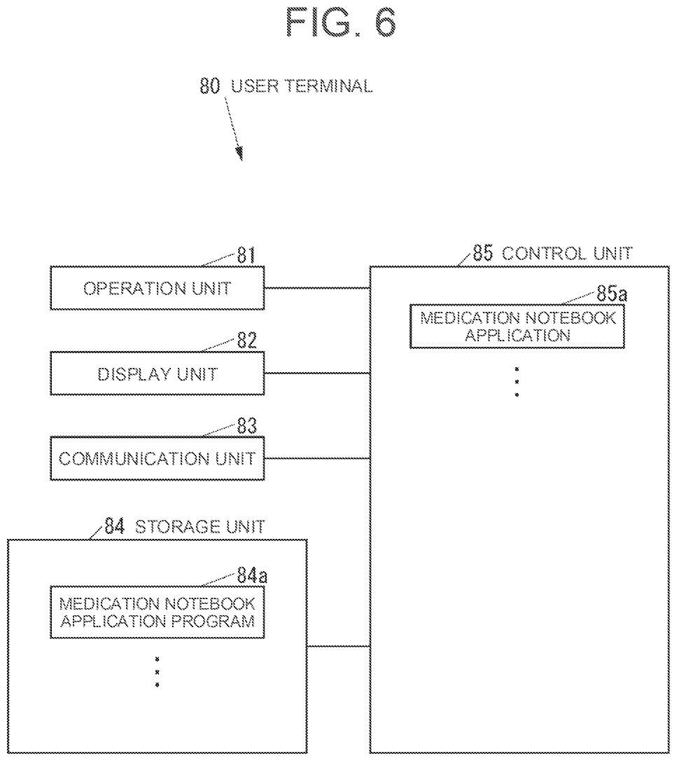

FIG. 6 is a block diagram of the user terminal 80.

As shown in FIG. 6, the user terminal 80 has an operation unit 81 which is an operation device such as, for example, a button into which various operations are input, a display unit 82 which is a display device such as, for example, an LCD for displaying various information, a communication unit 83 which is a communication device for communicating with an external device via a network such as a LAN or the Internet, or directly by wired or wireless means without going through the network, a storage unit 84 which is a non-volatile storage device such as, for example, a semiconductor memory or HDD for storing various information, and a control unit 85 which controls the entire user terminal 80.

The storage unit 84 can store a program (hereinafter referred to as “medication notebook application program”) 84a for an application (hereinafter referred to as “medication notebook application”) that sends dispensing reservation information to the dispensing reservation reception system. Other than the medication notebook application program 84a, the storage unit 65 can store the medication notebook application program for each pharmacy or each group of pharmacies. The medication notebook application is an application that allows a user to electronically execute the recording of dispensing to a patient. The medication notebook application program is released for each pharmacy or each group of pharmacies.

The control unit 85 has, for example, a CPU, a ROM that stores a program and various data, and a RAM as a memory used as a work area of the CPU. The CPU executes a program stored in the storage unit 84 or in the ROM.

The control unit 85 realizes the medication notebook application by executing the medication notebook application program. For example, the control unit 85 realizes the medication notebook application 85a by executing the medication notebook application program 84a.

The user terminal 80 operates as a BLE broadcaster. In other words, the control unit 85 periodically sends out an advertisement packet via the communication unit 83.

Next, the operation of the system 10 will be described.

First, the operation of the image forming device 40 when the specification conversion program is installed is described.

When a user, such as an administrator of the image forming device 40, wishes to install the specification conversion program on the image forming device 40, the user can store, in the USB memory, the specification conversion program which the user wishes to be installed on the image forming device 40, and then can attach the USB memory to the USB port 48 of the image forming device 40.

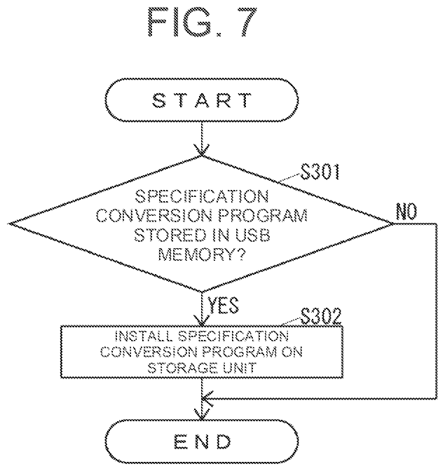

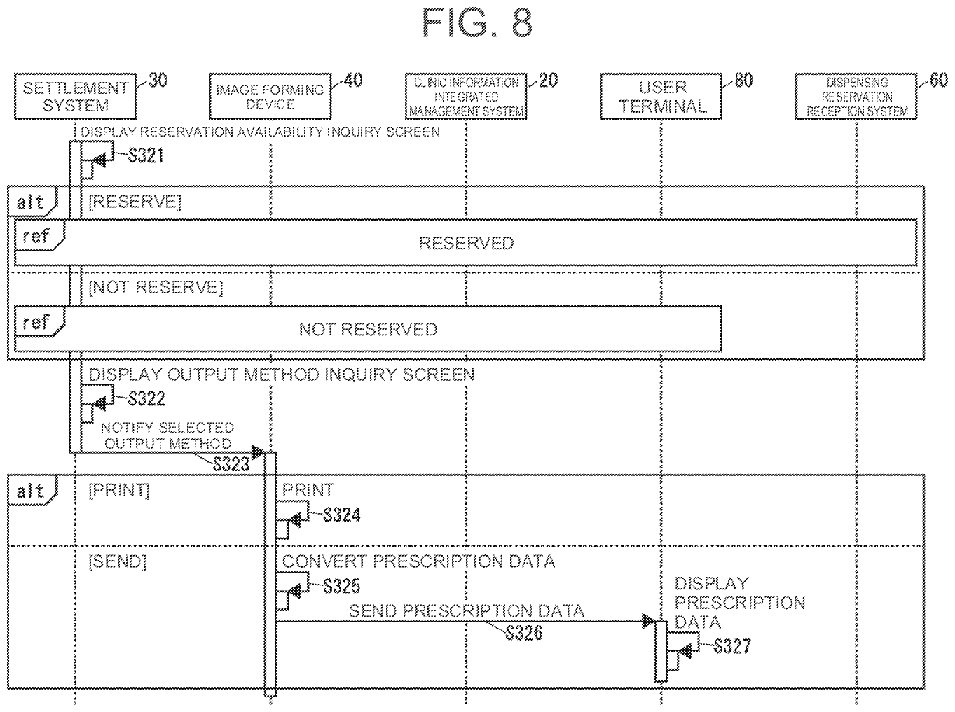

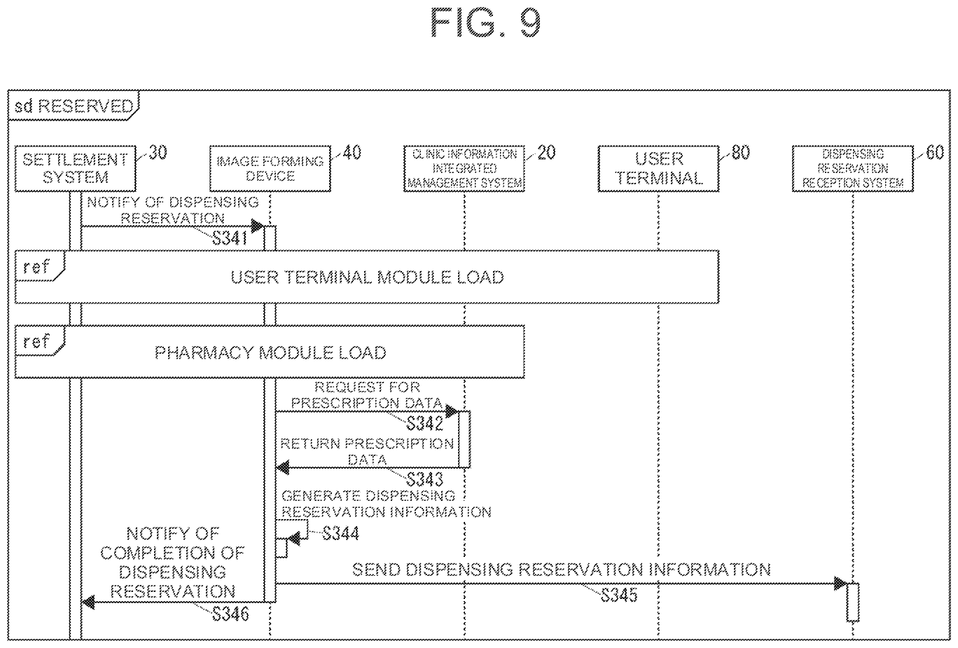

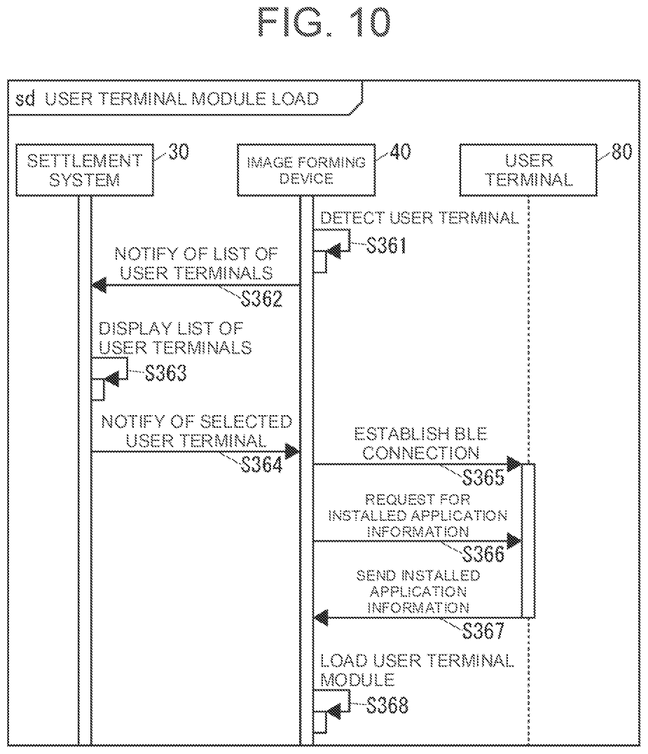

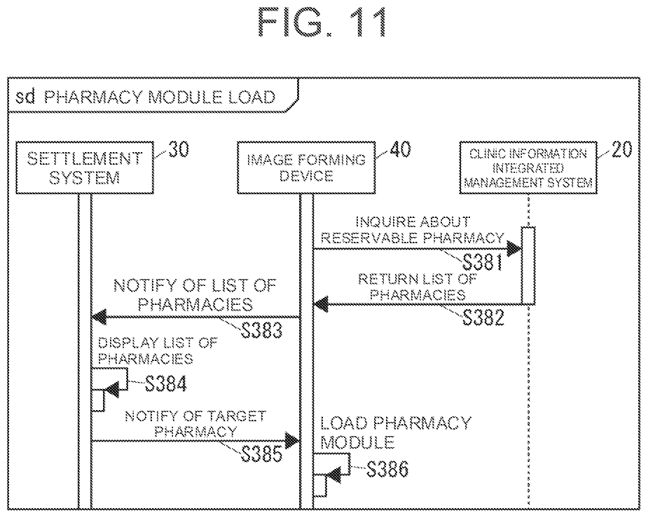

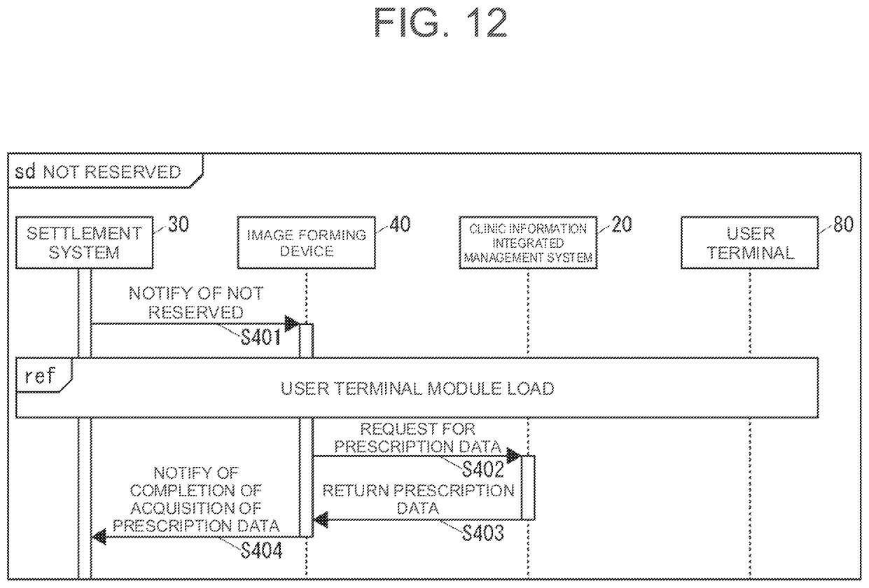

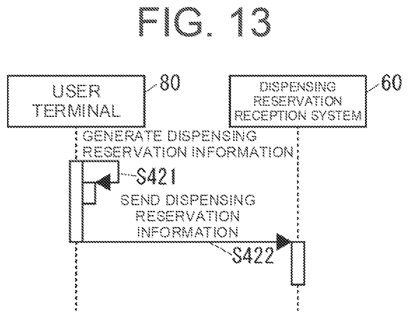

FIG. 7 is a flowchart of the operation of the image forming device 40 when the specification conversion program is to be installed.

When the USB memory is attached to the USB port 48 of the image forming device 40, the extension service management unit 190 of the image forming device 40 starts the module installation service. Upon being started, the module installation service executes the operation shown in FIG. 7.

As shown in FIG. 7, the module installation service determines whether or not the specification conversion program is stored in the USB memory attached to the USB port 48 (S301). Here, the module installation service accesses the USB memory via the extended application service IF unit 160, the platform service IF unit 130, the device operation service 123, the general-purpose OS 110, and the USB port 48, in that order.

When determining in 5301 that the specification conversion program is stored in the USB memory attached to the USB port 48, the module installation service installs the specification conversion program, which is stored in the USB memory, in an extended function service area of the storage unit 49 (S302). Here, the module installation service accesses the storage unit 49 via the extended application service IF unit 160, the platform service IF unit 130, the device operation service 123, and the general-purpose OS 110, in that order.

When determining in 5301 that the specification conversion program is not stored in the USB memory attached to the USB port 48, or when completing the process of S302, the module installation service completes the operation shown in FIG. 7.

Next, the operation of the system 10 when a medical expense is collected is described.

FIG. 8 is a sequence diagram of the operation of system 10 when the medical expense is collected. FIG. 9 is a sequence diagram of “Reserved” shown in FIG. 8. FIG. 10 is a sequence diagram of “User Terminal Module Load” shown in FIG. 9. FIG. 11 is a sequence diagram of “Pharmacy Module Load” shown in FIG. 9. FIG. 12 shows a sequence diagram for “Not Reserved” shown in FIG. 8.

When the medical expense is collected by the settlement unit 33, the control unit 36 of the settlement system 30 displays, on the display unit 32, a reservation availability inquiry screen as a screen to inquire whether or not to make a dispensing reservation at the pharmacy, as shown in FIGS. 8 to 12, if the prescription of the medicine is necessary (S321). Accordingly, a payer of medical expense such as a patient who is the subject of the paid medical expense (hereinafter referred to as the “subject patient”) or the like can select, via the operation unit 31 of the settlement system 30, whether or not to make a dispensing reservation at the pharmacy.

When the dispensing reservation at the pharmacy is selected in the reservation availability inquiry screen displayed in 5321, the control unit 36 of the settlement system 30 notifies the image forming device 40 of the dispensing reservation at the pharmacy (S341).

Upon receiving the notification in S341, the pharmacy reservation service of the image forming device 40 detects a connectable user terminal by scanning an advertisement packet sent by the user terminal (S361). Next, the pharmacy reservation service notifies the settlement system 30 of a list of user terminals detected in S361 (S362).

Upon receiving the notification in S362, the control unit 36 of the settlement system 30 displays, on the display unit 32, the list of the user terminals notified in S362 (S363). Accordingly, from among the list of user terminals displayed on the display unit 32, the payer of medical expense can select, via the operation unit 31 of the settlement system 30, the user terminal 80 owned by the payer itself.

When the user terminal is selected via the operation unit 31, the control unit 36 of the settlement system 30 notifies the image forming device 40 of the selected user terminal (S364).

Upon receiving the notification in S364, the pharmacy reservation service of the image forming device 40 sends a BLE connection request to the user terminal, among the user terminals detected in S361, that was notified in S364, and thereby establishes a BLE connection with this user terminal (S365). Hereinafter, it will be described on the condition that the user terminal by means of which the BLE connection is established with the image forming device 40 in S365 is the user terminal 80.

After the process of S365, via the BLE connection established in S365, the pharmacy reservation service requests the user terminal 80 for the information of the medication notebook application installed on the user terminal 80 (hereinafter referred to as “installed application information”) (S366).

Upon receiving the request in S366, the control unit 85 of the user terminal 80 sends the installed application information to the image forming device 40 via the BLE connection established in S365 (S367).

Upon receiving the installed application information sent in S367, the pharmacy reservation service of the image forming device 40, among the specification conversion modules installed on the image forming device 40, loads the specification conversion module (hereinafter referred to as “user terminal module”) that is compatible with to the medication notebook application indicated in the received installed application information (S368).

After the process of S368, the pharmacy reservation service inquires the clinic information integrated management system 20 about a reservable pharmacy with which the clinic that is compatible with the image forming device 40 is affiliated (S381).

Accordingly, based on the reservable pharmacy information 24a, the control unit 25 of the clinic information integrated management system 20 returns, to the image forming device 40, a list of reservable pharmacies with which the clinic that is compatible with the clinic information integrated management system 20 is affiliated (S382). The pharmacies indicated in the reservable pharmacy information can include a pharmacy that is not compatible with the medication notebook application installed on the user terminal 80.

Upon receiving the list of pharmacies returned in S382, the pharmacy reservation service of the image forming device 40 notifies the settlement system 30 of the received list of pharmacies (S383).

Upon receiving the notification in S383, the control unit 36 of the settlement system 30 displays, on the display unit 32, the list of pharmacies notified in S383 (S384). Accordingly, from among the list of pharmacies displayed on the display unit 32, the payer of medical expense can select, via the operation unit 31 of the settlement system 30, a pharmacy that the payer itself wishes to reserve for dispensing.

When a pharmacy is selected via the operation unit 31, the control unit 36 of the settlement system 30 notifies the image forming device 40 of the selected pharmacy (hereinafter referred to as the “target pharmacy” in the description of FIGS. 8 to 12) (S385).

Upon receiving the notification in S385, the pharmacy reservation service of the image forming device 40, among the specification conversion modules installed on the image forming device 40, loads the specification conversion module that is compatible with the medication notebook application of the target pharmacy notified in S385 (hereinafter referred to as the “pharmacy module”) (S386).

After the process of S386, the pharmacy reservation service of the image forming device 40 requests the clinic information integrated management system 20 for the prescription data of the target patient (S342).

Accordingly, the control unit 25 of the clinic information integrated management system 20 returns the prescription data of the target patient to the image forming device 40 (S343).

After the process of S343, the pharmacy module loaded in S386 generates the dispensing reservation information on the target pharmacy according to the specification of the medication notebook application of the target pharmacy (S344). Here, when being in need of sending the prescription data to the target pharmacy in order to make a dispensing reservation at the target pharmacy, the pharmacy module loaded in S386 includes, in the dispensing reservation information, the prescription data returned from the clinic information integrated management system 20 in S343. When including the prescription data in the dispensing reservation information, the pharmacy module loaded in S386 converts the prescription data into the specification of the medication notebook application of the target pharmacy.

After the process of S344, the pharmacy module loaded in S386 calls the API of the dispensing reservation reception system 60 of the target pharmacy to thereafter send, to the dispensing reservation reception system 60 of the target pharmacy, the dispensing reservation information generated in S344, in a sending method adapted to the specification of the medication notebook application of the target pharmacy (S345). In other words, the dispensing for the target patient is reserved at the target pharmacy.

After the process of S345, the pharmacy reservation service of the image forming device 40 notifies the settlement system 30 of the completion of the dispensing reservation (S346).

When it is selected on the reservation availability inquiry screen displayed in S321 that dispensing is not reserved to the pharmacy, the control unit 36 of the settlement system 30 notifies the image forming device 40 that the dispensing is not to be reserved to the pharmacy (S401).

When the pharmacy reservation service of the image forming device 40 receives the notification in S401, the system 10 loads the user terminal module by executing the processes of S361 to S368.

Next, the pharmacy reservation service of the image forming device 40 requests the clinic information integrated management system 20 for the prescription data of the target patient (S402).

Accordingly, the control unit 25 of the clinic information integrated management system 20 returns the prescription data of the target patient to the image forming device 40 (S403).

After the process of S403, the pharmacy reservation service of the image forming device 40 notifies the settlement system 30 of the completion of the acquisition of the prescription data (S404).

Upon receiving the notification in S346 or S404, the control unit 36 of the settlement system 30 displays, on the display unit 32, an output method inquiry screen as a screen to inquire about an output method of the prescription, i.e., whether to print the prescription on paper or to send the prescription to the medication notebook application (S322). Accordingly, the payer of the medical expense can select via the operation unit 31 of the settlement system 30 whether to print the prescription on paper or to send the prescription to the medication notebook application.

When the output method of the prescription is selected via the operation unit 31, the control unit 36 of the settlement system 30 notifies the image forming device 40 of the selected output method (S323).

When the image forming device 40 is notified in S323 from the settlement system 30 that the prescription is to be printed on paper, the pharmacy reservation service of the image forming device 40 prints the prescription by the printer 44 based on the prescription data returned from the clinic information integrated management system 20 in S343 or S403 (S324).

When the image forming device 40 is notified in S323 from the settlement system 30 that the prescription is to be sent to the medication notebook application, the user terminal module loaded in S368 converts the prescription data, which was returned from the clinic information integrated management system 20 in S343 or S403, into the specification of the medication notebook application installed on the user terminal 80 (S325).

After the process of S325, the user terminal module loaded in S368 sends, to the user terminal 80 via the BLE connection established in S365, the prescription data, whose specification has been converted in S325, in a sending method adapted to the specification of the medication notebook application installed on the user terminal 80 (S326).

When the prescription data is sent from the image forming device 40 to the user terminal 80 in S326, the medication notebook application of the user terminal 80 displays the prescription on the display unit 82 based on this prescription data (S327).

Next, the operation of the system 10 in the case of dispensing reservation by the medication notebook application will be described.

FIG. 13 is a sequence diagram of the operation of the system 10 dispensing reservation is made by the medication notebook application.

In the operations shown in FIG. 8 through FIG. 12, the dispensing is reserved by the image forming device 40. However, when the dispensing reservation reception system 60 of the pharmacy (hereinafter referred to as the “target pharmacy” in the description of FIG. 13) at which the patient wishes to make a dispensing reservation is compatible with the medication notebook application installed on the user terminal 80, it is also possible to make the dispensing reservation by the medication notebook application, as shown in FIG. 13.

As shown in FIG. 13, the medication notebook application of the user terminal 80 generates the dispensing reservation information on the target pharmacy according to the specification of the medication notebook application of the target pharmacy (S421). Here, when being in need of sending the prescription data to the target pharmacy in order to make the dispensing reservation at the target pharmacy, the medication notebook application of the user terminal 80 includes, in the dispensing reservation information, the prescription data sent from the image forming device 40 to the user terminal 80 in S326. For including the prescription data in the dispensing reservation information, the medication notebook application of the user terminal 80 converts the prescription data into the specification of the medication notebook application of the target pharmacy.

After the process of S421, the medication notebook application of the user terminal 80 calls the API of the dispensing reservation reception system 60 of the target pharmacy to thereafter send the dispensing reservation information, which is generated in S421, to the dispensing reservation reception system 60 of the target pharmacy (S422). That is, dispensing for the target patient is reserved at the target pharmacy.

As described above, the image forming device 40 can generate the dispensing reservation information in accordance with the specification of the medication notebook application of each of the plurality of pharmacies, and sends, to the dispensing reservation reception system of the target pharmacy, the dispensing reservation information generated in S344 in accordance with the specification of the medication notebook application of the target pharmacy as a pharmacy selected from among a plurality of pharmacies, in the sending method adapted to the specification of the medication notebook application of the target pharmacy (S345), thereby making it possible to make the dispensing reservation at the pharmacy, regardless of whether or not the user possesses the user terminal on which the medication notebook application that is compatible with the dispensing reservation reception system for accepting the dispensing reservation at the pharmacy by receiving the dispensing reservation information from the user terminal by the medication notebook application. Therefore, the user does not need to install, on the user terminal, the medication notebook application of the target pharmacy.

To the user terminal, the image forming device 40 sends (S326) the prescription data converted in S325 into the specification of the medication notebook application installed on the user terminal, in the sending method adapted to the specification of this medication notebook application. Therefore, regardless of the type of the medication notebook application installed on the user terminal, the image forming device 40 can display, in S327, the prescription on the medication notebook application installed on the user terminal. Therefore, the user can check the prescription via the medication notebook application by merely installing, on the user terminal, the medication notebook application of the user's preference.

Since the specification conversion module that generates the dispensing reservation information and converts the prescription data into the specification of the medication notebook application installed on the user terminal is a program different from the firmware 49b of the image forming device 40, the image forming device 40 can implement the addition of a new medication notebook application or implement a change in the specification of the already implemented medication notebook application by merely updating the specification conversion module (S302) without updating the firmware 49b. Accordingly, as compared to a configuration in which the firmware 49b of the image forming device 40 needs to be updated to implement the addition of the new medication notebook application or to implement the change in the specification of the already implemented medication notebook application, the image forming device 40 can shorten the development period of software to implement the addition of the new medication notebook application or to implement the change in the specification of the already implemented medication notebook application.

In this embodiment, the image forming device 40 installs the specification conversion program when the USB memory (flash drive) in which the specification conversion program is stored is attached to the USB port 48. However, the image forming device 40 may install the specification conversion program by any other method. For example, the image forming device 40 may install this specification conversion program when a storage device, other than the USB memory, in which the specification conversion program is stored is connected.

The communication between the image forming device 40 and the user terminal is realized by the BLE, in this embodiment. However, the communication between the image forming device 40 and the user terminal may be realized by a communication method other than the BLE.

Any of the plurality of programs in this embodiment may be configured as a single program. For example, the extension program 49c and the extension service program may be configured as a single program.

Although the electronic device of the present disclosure is an image forming device in the present embodiment, the electronic device can also be an electronic device other than the image forming device, such as, for example, a PC (Personal Computer).

What is claimed is:

1. An electronic device that receives dispensing reservation information as information of a dispensing reservation at a pharmacy and thereby sends the dispensing reservation information to a dispensing reservation reception system that receives the dispensing reservation at the pharmacy,
wherein
the dispensing reservation reception system is capable of receiving the dispensing reservation information sent by a medication notebook application of a user terminal that realizes the medication notebook application as an application for sending the dispensing reservation information, and
the electronic device is capable of generating the dispensing reservation information in accordance with a specification of the medication notebook application of each of a plurality of the pharmacies, and sends, to the dispensing reservation reception system of a target pharmacy, the dispensing reservation information generated in accordance with the specification of the medication notebook application of the target pharmacy as a pharmacy selected from among the plurality of the pharmacies, in a sending method adapted to the specification of the medication notebook application of the target pharmacy.

2. The electronic device according to claim 1, wherein
the electronic device is provided with an extension service that provides a function other than a standard function of the electronic device,
a program for the extension service is a program different from a firmware of the electronic device, and
the electronic device generates the dispensing reservation information by a specification conversion module which is the extension service for converting a specification of the dispensing reservation at the pharmacy.

3. The electronic device according to claim 1, wherein
the electronic device acquires the information of the medication notebook application installed on the user terminal, converts prescription data as data of prescription into the specification of the medication notebook application shown in the acquired information, and sends, to the user terminal, the prescription data with the converted specification, in the sending method adapted to the specification of the medication notebook application.

4. The electronic device according to claim 3, wherein
the electronic device is provided with an extension service that provides a function other than a standard function of the electronic device,
a program for the extension service is a program different from a firmware of the electronic device, and
the electronic device converts the specification of the prescription data by a specification conversion module which is the extension service for converting a specification of the dispensing reservation at the pharmacy.

5. A computer-readable non-transitory recording medium storing a pharmacy reservation program that receives dispensing reservation information as information of a dispensing reservation at a pharmacy and thereby sends the dispensing reservation information to a dispensing reservation reception system that receives the dispensing reservation at the pharmacy,
wherein
the dispensing reservation reception system is capable of receiving the dispensing reservation information sent by a medication notebook application of a user terminal that realizes the medication notebook application as an application for sending the dispensing reservation information, and
the pharmacy reservation program is capable of causing an electronic device to generate the dispensing reservation information in accordance with a specification of the medication notebook application of each of a plurality of the pharmacies, and causes the electronic device to send, to the dispensing reservation reception system of a target pharmacy, the dispensing reservation information which the pharmacy reservation program caused the electronic device to generate in accordance with the specification of the medication notebook application of the target pharmacy as a pharmacy selected from among the plurality of the pharmacies, in a sending method adapted to the specification of the medication notebook application of the target pharmacy.

6. A pharmacy reservation system that receives dispensing reservation information as information of a dispensing reservation at a pharmacy and thereby sends the dispensing reservation information to a dispensing reservation reception system that receives the dispensing reservation at the pharmacy,
wherein
the dispensing reservation reception system is capable of receiving the dispensing reservation information sent by a medication notebook application of a user terminal that realizes the medication notebook application as an application for sending the dispensing reservation information, and
the pharmacy reservation system is capable of generating the dispensing reservation information in accordance with a specification of the medication notebook application of each of a plurality of the pharmacies, and sends, to the dispensing reservation reception system of a target pharmacy, the dispensing reservation information generated in accordance with the specification of the medication notebook application of the target pharmacy as a pharmacy selected from among the plurality of pharmacies, in a sending method adapted to the specification of the medication notebook application of the target pharmacy.
